(19) United States
(12) Patent Application Publication
(10) Pub. No.: US 2023/0051451 A1
(43) Pub. Date: Feb. 16, 2023

(54) INDOOR AIRFLOW CONTROL SYSTEM

(71) Applicant: WEMOTE TECHNOLOGIES LIMITED, Hong Kong (CN)

(72) Inventors: YAU SANG STEPHEN CHENG, Hong Kong (CN); Wing Shing Andy Choi, Hong Kong (CN)

(21) Appl. No.: 17/967,883

(22) Filed: Oct. 17, 2022

Related U.S. Application Data
(63) Continuation of application No. PCT/CN2022/074339, filed on Jan. 27, 2022.
(60) Provisional application No. 63/148,111, filed on Feb. 10, 2021.

(51) Int. Cl. F24F 8/167; F24F 8/95; F24F 8/192; F24F 7/06

(52) U.S. Cl. CPC F24F 8/167; F24F 8/95; F24F 8/192; F24F 7/06

(57) ABSTRACT

A system for airflow control includes at least two apparatus. The apparatus configures to create the required airflow to direct, remove and deactivate pathogenic aerosols in the air by working together. The apparatus can equip with low power fan.

### TECHNICAL FIELD

The present invention relates to air purifier, and, in particular embodiments, to an airflow control system apparatus for eliminating pathogens.

### BACKGROUND

Covid-19 has raised the awareness that indoor air can always contain pathogenic aerosols. It would be desirable to provide an apparatus and/or a method for purifying the indoor air by the means of trapping the pathogenic aerosols, in order to improve public health and safety.

As shown in FIG. 1, there are several typical solutions to keep the indoor air clean, such as opening the window for more rapid air replacement with fresh air, deploying one or more air disinfection machines at various locations, applying coatings onto surfaces or walls which can deactivate or kill pathogens or using UV light to kill nearby pathogens.

Unfortunately, in the typical solution describe as below, the pathogenic aerosols at locations X may linger in the air for considerable amount of time before being deactivated or removed. While they linger in the air, many healthy people can be infected.

In addition, typical portable air clean device (i.e., air purifier) based on the blockage-type removal mechanism and usually requires strong force to move the air through the filter or the like, resulting in devices being more complex, heavier and more expensive.

### SUMMARY

In particular embodiments, an airflow control system may prevent the pathogenic aerosols to linger indoor for considerable amount of time and maintain the indoor air clean.

In accordance with an embodiment, the airflow control system includes at least two apparatus. The apparatus configures to create the required airflow to direct, remove and deactivate pathogenic aerosols in the air by working together.

Optionally, the apparatus includes a trapping component configured to trap pathogenic aerosols in the air and an airflow generator configured to blow or suck the air to create the required airflow.

Optionally, the trapping component includes: a corridor with two open ends and several partitions arranged in the corridor to separate the corridor into several narrow channels.

An advantage of a preferred embodiment of the present disclosure is that the pathogenic aerosols have much higher chance to be attached and remained on the surface of the partition and the airflow remains unrestricted at the same time.

Optionally, the narrow channel is winding.

Optionally, the airflow generator is a low-power fan within the apparatus.

Optionally, the apparatus further includes: a dust preventor positioned on one end of the corridor.

Optionally, at least a part of a surface of the several narrow channels with disinfecting properties.

Optionally, the surface with disinfecting properties includes: a top layer configured to retain the pathogenic aerosols and a lighting device located under the top layer and configured to provide light.

Optionally, a surface of the top layer is rough.

Optionally, the top layer contains light activated photocatalyst.

Optionally, the light activated photocatalyst includes: zinc oxide, g-C₃N₄ (graphitic carbon nitride) or titanium dioxide.

Optionally, the lighting device is made by TFEL or organic LED.

Optionally, at least a part of an inner surface of the trapping component having a static charge as an inherent property of the material, as a result of static charge generated by air friction or an applied electric field.

Optionally, the trapping component is filter.

Optionally, the airflow generator is an external compressor connected to the apparatus via hose or ducts.

Optionally, the airflow generator is a fan within the apparatus.

Optionally, the apparatus further includes: air intake for the air getting into the apparatus.

An advantage of a preferred embodiment of the present disclosure is providing a system for generating required airflow to direct, remove and deactivate pathogenic aerosols in the indoor air and an apparatus with lower power fan.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure, and the advantages thereof, reference is now made to the following descriptions taken in conjunction with the accompanying drawings, in which:

FIG. 1 illustrates typical methods for disinfection of air in indoor environment; the source of FIG. 1 is “How can airborne transmission of COVID-19 be minimized?”, Environmental International 142 (2020) 105832, and the label “X” is not in the source illustration;

FIG. 2a illustrates schematic diagrams of airflow control system in accordance with various embodiments of the present disclosure to address gaps not met by current methods;

FIG. 2b illustrates schematic diagrams of airflow control system in accordance with preferred embodiments of the present disclosure;

FIG. 3a illustrates schematic diagrams of the apparatus arrange in the indoor place in accordance with various embodiments of the present disclosure; and

FIG. 3b illustrates cross-section diagram of the first apparatus and the second apparatus shown in FIG. 3a in accordance with various embodiments of the present disclosure.

FIG. 4a illustrates cross-section diagram of the typical air purifier.

FIG. 4b illustrates schematic diagrams of the corridor in accordance with various embodiments of the present disclosure.

FIG. 4c illustrates schematic diagrams of the corridor in accordance with preferred embodiments of the present disclosure.

FIG. 5 illustrates schematic diagrams of the trapping component in accordance with various embodiments of the present disclosure.

FIG. 6 illustrates schematic diagrams of the surface having disinfecting properties in accordance with various embodiments of the present disclosure.

FIG. 7a illustrates schematic diagrams of the comparative example in accordance with various embodiments of the present disclosure.

FIG. 7b illustrates schematic diagrams of inventive example 1 in accordance with various embodiments of the present disclosure.

FIG. 7c illustrates schematic diagrams of inventive example 2 in accordance with various embodiments of the present disclosure.

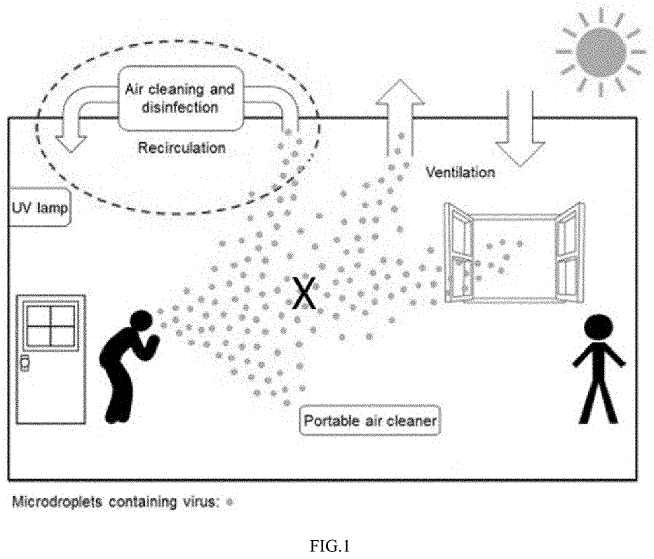

Corresponding numerals and symbols in the different figures generally refer to corresponding parts unless otherwise indicated. The figures are drawn to clearly illustrate the relevant aspects of the various embodiments and are not necessarily drawn to scale.

### DETAILED DESCRIPTION OF ILLUSTRATIVE EMBODIMENTS

The making and using of the presently preferred embodiments are discussed in detail below. It should be appreciated, however, that the present invention provides many applicable inventive concepts that can be embodied in a wide variety of specific contexts. The specific embodiments discussed are merely illustrative of specific ways to make and use the disclosure, and do not limit the scope of the disclosure.

The present disclosure will be described with respect to preferred embodiments in a specific context, namely air refresh system applied to eliminate the pathogenic aerosols. The air refresh system includes at least two apparatus configured to provide the required airflow. The invention may also be applied, however, to a variety of environments. Hereinafter, various embodiments will be explained in detail with reference to the accompanying drawings.

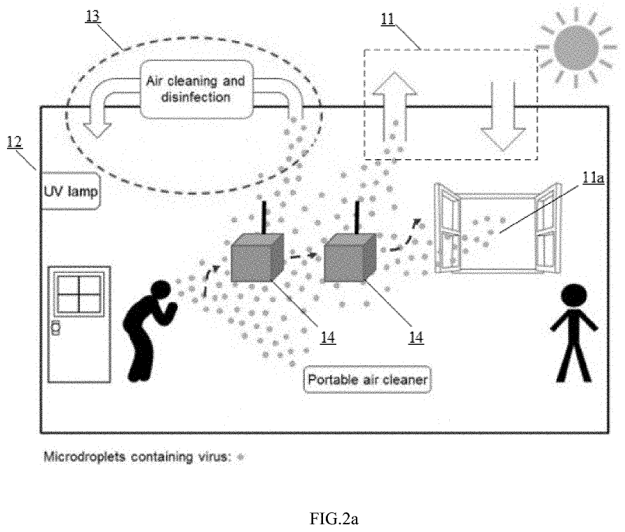

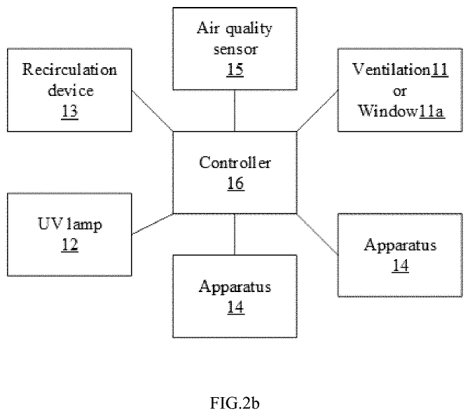

FIG. 2a illustrates an air refresh system in accordance with various embodiments of the present disclosure. The existing air refresh system includes a ventilation 11, a UV lamp 12, a recirculation device 13, but do not address some of the aerosols lingering in the air. Addition of least two apparatus 14 addresses this gap by trapping pathogenic aerosols.

As shown in FIG. 2a, the apparatus 14 can be placed in the indoor space to provide the required airflow. The movement of the indoor air could effectively reduce the linger time of the pathogenic aerosols.

In some embodiments, as shown in FIG. 2b, the air refresh system may further include an air quality sensor 15 and a controller 16. The controller 16 can be implemented by a smart terminal, personal computer, server or the like. The air quality sensor 15 could be any suitable type of sensor for detecting the presence or concentration of any number of target pollutants.

The controller 16 is connected with all the components of the air refresh system by wired/wireless network and configured to control the operation of the apparatus 14 to direct air towards the ventilation 11, UV lamp 12, recirculation device 13 or window 11a according to the air quality detected by the sensor. For instance, the controller 16 may activate the apparatus 14 and the UV lamp when the concentration of pathogenic aerosols detected by the sensor 15 is over the predetermined upper limit.

It should be noted that the components of the air refresh system could be omitted in some embodiments and the other components could be added in the air refresh system according to the needs of the actual situation, but not limit to the components shown in FIG. 2a and FIG. 2b.

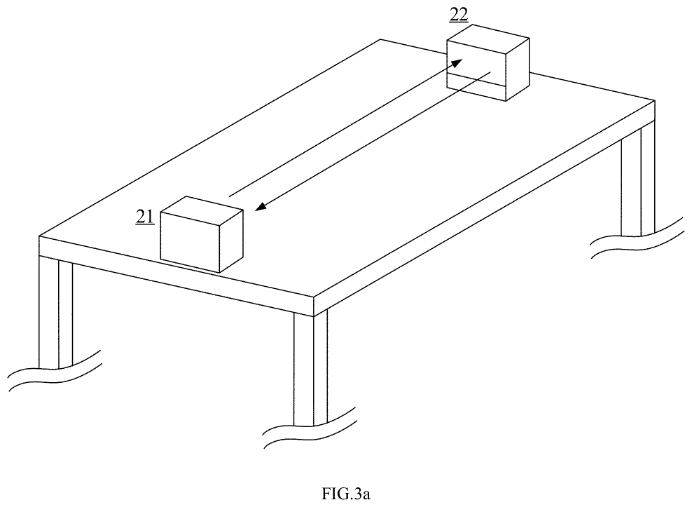

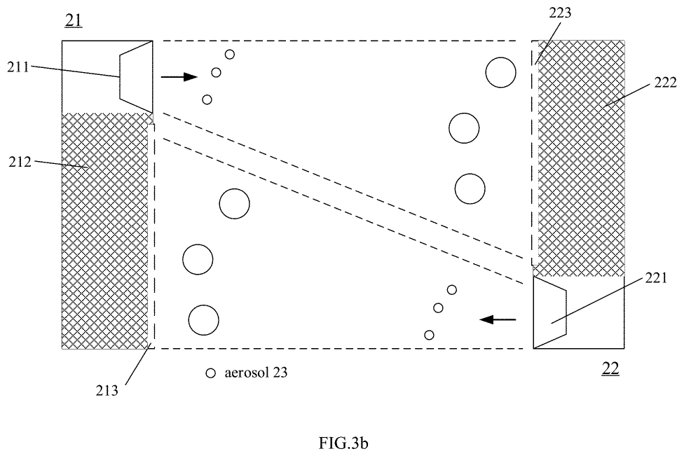

FIG. 3a illustrates an air refresh system in accordance with various embodiments of the present disclosure. As shown in FIG. 3a, the air refresh system is consisted of the first apparatus 21 and the second apparatus 22. The first apparatus 21 and the second apparatus 22 are correspondingly arranged (i.e., placed at the opposite ends of the table).

Generally, people will take off their masks when they are in the indoor place, such as the meeting room, home or the restaurant. The first apparatus 21 and second apparatus 22 can move the air (the direction of the airflow is shown as the arrow in FIG. 3a) to help to create air movement between the apparatus, which would sweep nearby pathogenic aerosols into either apparatus, effectively reducing the time the pathogens lingering in the area.

FIG. 3b illustrates the structural diagram of the first and second apparatus in accordance with various embodiments of the present disclosure. As shown in FIG. 3b, the first apparatus 21 includes an airflow generator source 211, a trapping component 212 and an air intake 213. The second apparatus 22 also includes an airflow generator 221, a trapping component 222 and an air intake 223.

The airflow generator (211,221) is any suitable mechanical component for generating the required airflow, and are typically electrically powered. The electricity can come from chemical batteries, solar cells, electrical outlets via wires and cables or wirelessly transmitted. In some embodiments, the airflow generator can be a fan which is component built into the trapping component 212. Alternatively, the airflow generator can be an air compressor as an external source providing airflow.

The trapping component (212,222) is configured to trap the pathogenic aerosols when the indoor air passes the component. In one embodiment, the trapping component can be a filter. In the preferred embodiment, the trapping component can be an open-channel tortuous path construction, such as a zig-zag trap. Optionally, trapping ability can be further improved when the surface of the trapping component has a static charge as an inherent property of the material, as a result of static charge generated by air friction or an applied electric field.

The air intake (213,223) is with any suitable size and shape. The size, shape and the structure of the air intake can be determined according to the needs of the actual operating situation.

In operation, refer to FIG. 3b, the air on the upper-level moves from the first apparatus to the second apparatus and the air on the under-level moves from the second apparatus to the first apparatus. The aerosol 23 gets blown towards the direction of the airflow. When the air reaches the opposite apparatus, the air will be taken by the air intake and then pass through the trapping component. The aerosol in the air could be trap in the trapping component. In this way, the pathogenic aerosol in the indoor air could be removed quickly.

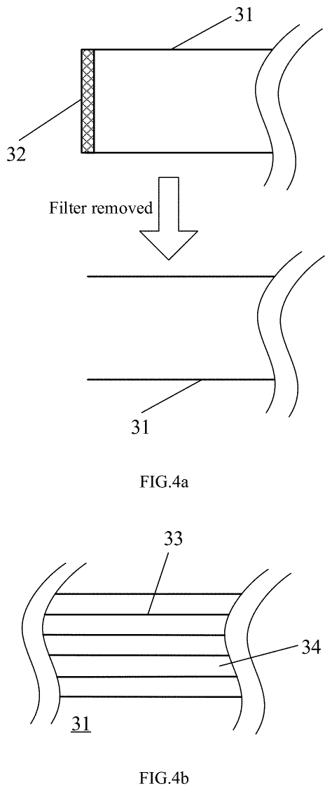

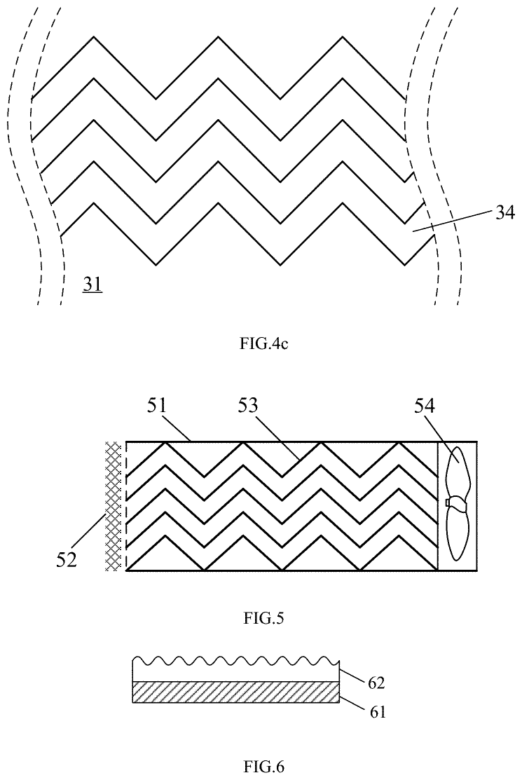

FIG. 4a illustrates a cross-section diagram of the typical air purifier. As shown in FIG. 4a, the air purifier includes a corridor 31 and a filter 32 installed in the corridor 31. The filter 32 can be any suitable type of the air filter, such as high efficiency particulate air (HEPA) filter.

Because of the strong resistance of the filter, the air purifier is required to equip a more powerful fan to create strong airflow through the filter. Alternatively, the filter of the air purifier can be removed and a lower power fan can be employed in the air purifier. But the pathogens would not be trapped in this situation.

FIG. 4b illustrates the structural diagram of the corridor in accordance with various embodiments of the present disclosure. The corridor can equip with low-power fan while the filter is removed. As shown in FIG. 4b, several partition 33 arrange in the corridor 31 and separate the corridor 31 into several narrower channels 34.

The specific width of the narrower channel can be determined by those skilled in the art. It can be any suitable size that is significant narrower the channel of the traditional air purifier based on blockage-type removal mechanism.

An advantage of the corridor 31 with several partition 33 shown in FIG. 4b is that the pathogenic aerosols have much higher chance to be attached and remained on the surface of the partition 33 and the airflow remains unrestricted at the same time.

In preferred embodiments, as shown in FIG. 4c, the narrower channel 34 can be a tortuous path of the air. The tortuous path 34 can cause an air turbulence which is help to increase the chance of the pathogen aerosols to hit and be retained on the surface of the partition 33.

Although the tortuous path is created by Zig-zagging structure shown in FIG. 4c, those skilled in the art could use other structures or ways to create the tortuous path. For instance, the spiral pathways can also be used to create the tortuous path.

FIG. 5 illustrates a trapping component in accordance with various embodiments of the present disclosure. As shown in FIG. 5, the trapping component includes a corridor 51, a dust preventor 52, several partition 53 and a low-powered fan 54.

The corridor 51 is surround by the side wall and with two open ends. The partition 53 is arrange in the channel to separate the corridor 51 into several tortuous narrower channels.

The dust preventor 52 is positioned on one end of the channel to prevent the dust in air from entering into the channel. In some embodiments, the dust preventor 52 can be a coarse mesh or a filter. The low-powered fan 54 is positioned on the other end of the channel to pull air through the channels.

In operation, the low-powered fan 54 drive the air into the corridor 51. Then the air turbulence is created by the tortuous narrower channels. When the air turbulence through the corridor 51, the pathogen aerosols will hit and retain on the surface of the partition. The width of the channel is made narrow enough such that over the course of the air movement through the channel, pathogenic aerosols bump into the corridor walls and become attached or immobilized. Alternatively, the position of the low-powered fan 54 can be changed and configured to pull the air into the corridor.

It should be noted that the components of the trapping component such as the dust preventor 52, could be omitted in some embodiments and the other components could be added according to the needs of the actual situation, but not limit to the components shown in FIG. 5.

In some embodiments, the surface of the channel is made to have disinfecting properties. FIG. 6 illustrates the structural diagram of the surface having disinfecting properties in accordance with various embodiments of the present disclosure.

As shown in FIG. 6. the partition includes a light device 61 and the surface 62 of the light device is used as the surface of the channel. The surface 62 of the lighting device 61 is modified to be retain the pathogen (for example, having a rough surface) or have a layer of material to do so. In the case of TFEL (thin-film electroluminescent), the flat lighting can be bent without adverse effect of the device, and can easily be used to create the tortuous path to trap the pathogenic aerosol more effective.

In some embodiments, the surface 62 of the lighting device 61 contains photocatalyst such that the pathogens on the surface can be deactivated by the photocatalyst when light is provided by the lighting device.

One advantageous feature of the surface having disinfecting properties shown in FIG. 6 is that the virous or bacteria in the pathogens can be inactivated.

In order to demonstrate the validity and illustrate the effect of the system and apparatus according to the embodiment, several experiments are providing and detail described as follow.

Experiment 1:

Material Used:

1) Water containing green fluorescent paint (made from a water-soluble paste diluted to 5% concentration with water);

2) UV lamp (5 w, 395 nm) powered by 5V USB power source;

3) 0.5 mm thick cardboard (rough surface of paper fibers which allow retention of aerosols);

4) Adhesive tape, silicone sealant (to seal the zig zag configuration to prevent leakage);

5) 5 cm diameter fan powered by 5V USB power source with approximately 1.2 L/min air movement capacity when unobstructed;

6) Small Hand-Held Sonic Humidifier.

Models of the trapping component are built by 0.5 mm thick cardboard. The low-powered fan is used to blow or suck air into the corridor. A sonic humidifier is used to generate micron and sub-micron water aerosols, and fluorescent dye is added into the water to determine if the aerosols (exposed by UV-A light at 395 nm wavelength) were trapped within the models.

### Comparative Example

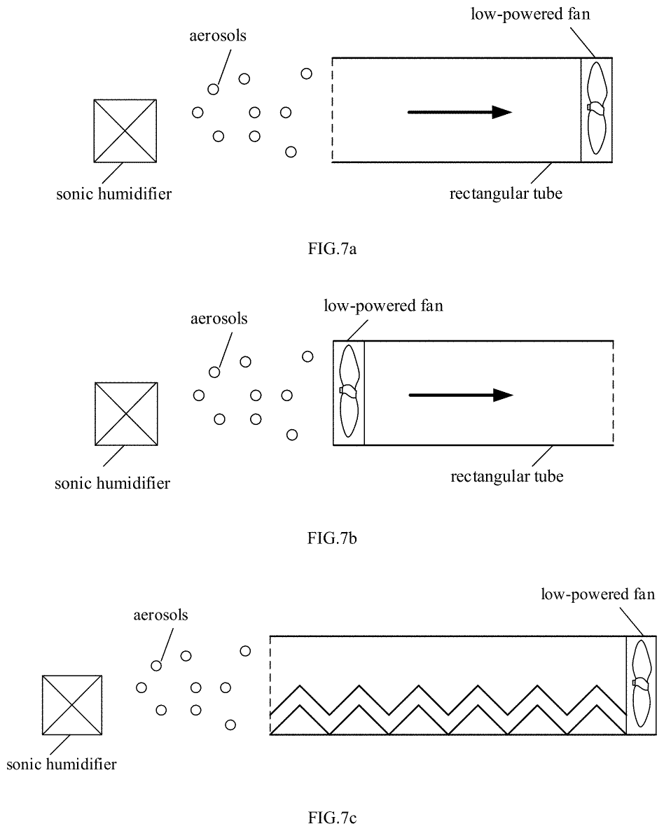

As shown in FIG. 7a, a rectangular tube with a 5×5 cm square opening of 20 cm length is created from 0.5 mm thick cardboard (the length of the corridor as 18 cm). 0.5 cm path was created by blocking the rest of the tube, which allow air to flow into this channel. The fan was fitted at the other end of the tube, with the air blowing outwards, thus, air was drawn into the tube at the air intake opening.

Aerosol particles containing fluorescent dye is generated by the sonic humidifier, and placed 10 cm away from the air intake opening of the tube. The stream of aerosols was aimed directly at the opening for 60 seconds.

The result of the comparative example shows that the fluorescent markers uniformly across the entire 20 cm length.

### Inventive Example 1

As shown in FIG. 7b, the location of the fan has been changed to the air intake opening, such that the air turbulence is increase.

The result of the inventive example 1 shows that fluorescence is observed mostly within 1-2 cm from the opening and grow fainter inwards, after 60 seconds of aerosol directed at the fan. No fluorescence is observed beyond 9 cm into the tube.

### Inventive Example 2

As shown in FIG. 7c, there is a winding path to create the air turbulence. The length of the tube is 10 cm. The length of path is 18 cm and the parallel distance of the channel width is 0.5 cm.

The result of the inventive example 2 shows that almost all the fluorescence is concentrated on the first bend and the last trace of fluorescence is detected on the 3^{rd} bend, after 60 seconds of aerosol directed at the fan.

The comparative example shows that with little turbulence, aerosol particle can travel through the narrow corridor and reach the end. The inventive example 1 shows that by increasing the turbulence of the air entering the corridor, the aerosol particle becomes stuck onto the walls and are not detected at the end of the corridor. The inventive example 2 shows that turbulence can also be created by winding paths designed to force the constant change of airflow direction within the corridors. Aerosol particles in such a situation also become stuck onto the walls and are not detected at the end of the corridor.

Experiment 2:

Material Used:

1) thin-film electroluminescent materials (TFEL) lighting panel (white colored light of 10×10 cm area, powered by 5V USB) of 100 lumens brightness according to supplier;

2) ZnO photocatalyst from Syn-Tech Fuel Management & Technology Co., Ltd;

3) TiO₂ photocatalyst from Sambo Tech and Titanology;

4) g-C₃N₄ photocatalyst from China National Petroleum Corporation;

5) Household 3-ply facial tissue paper;

6) Agar dish (white agar) to grow bacteria.

Model of the surface with disinfecting properties is made by the TFEL lighting panel and treated with various photocatalysts.

All three photocatalysts are in the form of water-based solutions, which are sprayed onto a surface and allowed to try. Bacteria are abundant in our environment. The experiment 2 is to find one which does not die naturally at the same rate as when a photocatalyst is present.

When the thin form-factor lighting with disinfected surface is applied in practice, it would likely (but not necessarily) have a fibrous surface (i.e., covered with same type of fibers as those used to create HEPA filters). As a simulation, 1-ply of tissue paper is used instead and placed directly onto the surface. Each photocatalyst was sprayed once evenly onto an individual ply and allowed to air-dry for 24 hours.

Bacteria in the kitchen sink is collected by wiping with a damp cloth, and draining the water into a spray bottle, and used immediately. Bacteria would be applied onto the tissue paper by spraying evenly once over the surface, and allowed to air-dry for 1 hour.

All tissue samples would be placed in the dark, except those examples which would be put directly onto the surface of the TFEL lighting panel for light exposure. The tissue paper is not adhered onto the surface as it would make preparation of the agar plates much easier.

After exposure for 22 hours, tissue paper of approximately 2×2 cm is put into direct contact with agar surface for 5 seconds. The bacteria would be observed after around 32 hours.

The result of different sample detail described on the table as follow:

| | | | light | Result of bacteria |
|---|---|---|---|---|
| sample | photocatalyst | Bacteria | exposure | growing on agar plate |
| Baseline | None | None | None | 1 colony |
| Control | None | Yes | None | Numerous (>50) |
| Sample 1 | ZnO | Yes | None | Similar to control |
| Sample 2 | ZnO | Yes | Yes | Around 10 colonies, much |
| | | | | lower density vs Sample 1 |
| Sample 3 | g-C₃N₄ | Yes | None | Similar to Control |
| Sample 4 | g-C₃N₄ | Yes | Yes | 90% in terms of density |
| | | | | vs Sample 3 |
| Sample 5 | TiO₂ | Yes | None | Numerous, higher density |
| | | | | than control but colonies |
| | | | | are much smaller in size |
| Sample 6 | TiO₂ | Yes | Yes | Similar to Sample 5 |

The baseline sample is the tissue paper without bacteria. The control sample is the tissue with bacteria.

According to the table, the baseline sample shows that there is minimal contamination in the test environment. Effectiveness is compared between control sample which has no photocatalyst. Sample 1, 3 and 5 have photocatalyst but without light activation. Sample 2, 4 and 6 have photocatalyst activated by the light.

According the result comparison, the ZnO photocatalyst has significant disinfection property, while g-C₃N₄ shows minor effect, and TiO₂ exhibits little difference.

The light exposure can be integrated with the photocatalyst to create a simple and versatile disinfection article. In particular, the light source made by TFEL can be bent, cut and shaped into different many desired forms for further incorporation into useful final products.

Although embodiments of the present invention and its advantages have been described in detail, it should be understood that various changes, substitutions and alterations can be made herein without departing from the spirit and scope of the invention as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An airflow control system, comprising at least two apparatus (14) configured to work together to create a required airflow.

2. The airflow control system according to claim 1, wherein the apparatus (14) comprises:
a trapping component (212) configured to trap pathogenic aerosols in the air;
an airflow generator (211) configured to blow or suck the air to create the required airflow.

3. The airflow control system according to claim 2, wherein the trapping component (212) comprises:
a corridor (31) with two open ends;
several partitions (33) arranged in the corridor (31) to separate the corridor (31) into several narrow channels (34).

4. The airflow control system according to claim 3, wherein the narrow channel (34) is winding.

5. The airflow control system according to claim 3, wherein the airflow generator (211) is a low-power fan within the apparatus.

6. The airflow control system according to claim 3, wherein the apparatus further comprises: a dust preventor (52) positioned on one end of the corridor.

7. The airflow control system according to claim 3, at least a part of a surface of the several narrow channels with disinfecting properties.

8. The airflow control system according to claim 7, wherein the surface with disinfecting properties comprises:
a top layer (62) configured to retain the pathogenic aerosols;
a lighting device (61) located under the top layer and configured to provide light.

9. The airflow control system according to claim 8, wherein a surface of the top layer (62) is rough.

10. The airflow control system according to claim 8, wherein the top layer (62) contains light activated photocatalyst.

11. The airflow control system according to claim 10, wherein the light activated photocatalyst comprises: zinc oxide, g-C₃N₄ (graphitic carbon nitride) or titanium dioxide.

12. The airflow control system according to claim 7, wherein the lighting device (61) is made by TFEL or organic LED.

13. The airflow control system according to claim 2, wherein at least a part of an inner surface of the trapping component having a static charge as an inherent property of the material, as a result of static charge generated by air friction or an applied electric field.

14. The airflow control system according to claim 2, wherein the trapping component (52) is filter.

15. The airflow control system according to claim 2, wherein the airflow generator (54) is an external compressor connected to the apparatus via hose or ducts.

16. The airflow control system according to claim 2, wherein the airflow generator (54) is a fan within the apparatus.

17. The airflow control system according to claim 2, wherein the apparatus further comprises: air intake (213) for the air getting into the apparatus.
